(19) United States
(12) Patent Application Publication
(10) Pub. No.: US 2020/0407691 A1
(43) Pub. Date: Dec. 31, 2020

(54) Methods and Materials for Modulation of Cell Secretome Production and Composition

(71) Applicant: UNIVERSITY OF SOUTH CAROLINA, Columbia, SC (US)

(72) Inventors: EHSAN JABBARZADEH, Columbia, SC (US); Maria Yanez, West Columbia, SC (US); Sara Eslambolchimoghadam, Columbia, SC (US)

(21) Appl. No.: 16/857,932

(22) Filed: Apr. 24, 2020

Related U.S. Application Data
(60) Provisional application No. 62/867,541, filed on Jun. 27, 2019.

(51) Int. Cl. C12N 5/0775; C12N 5/00; A61K 35/28

(52) U.S. Cl. CPC C12N 5/0668; C12N 5/0018; C12N 2500/76; C12N 2500/38; C12N 2500/30; A61K 35/28

(57) ABSTRACT

Methods for culturing cells, compositions comprising the cell culture product, and applications for the compositions are described. Methods include culturing cells with a media that includes a catalyst compound, which can include a stilbene or a nicotinamide compound, so as to control the secretome of the cells. Compositions that include the cell culture product include the cell secretome or components thereof in conjunction with at least one catalyst compound. Products can be utilized as a cell culture media or can be utilized in therapeutic applications.

### CROSS REFERENCE TO RELATED APPLICATION

This application claims filing benefit of U.S. Provisional Patent Application Ser. No. 62/271,019, having a filing date of Jun. 27, 2019, which is incorporated herein by reference in its entirety.

### FEDERAL RESEARCH STATEMENT

This invention was made with government support under Grant No. 1811949, awarded by the National Science Foundation, and under Grant No. R03AR063338, awarded by the National Institutes of Health. The Government has certain rights in the invention.

### BACKGROUND

Cells communicate and exchange information through contact and secreted biologically active substances. The product of cellular secretion is referred to as secretome, which is composed of soluble and insoluble factors containing proteins, cytokines, growth factors, extracellular matrix protein, and extracellular vesicles, among others. The proteins of the secretome play essential roles in the regulation of many physiological processes via paracrine/autocrine mechanisms, and they are of increasing interest as potential biomarkers and therapeutic targets in diseases.

Proteins released by cells into conditioned media in vitro have been studied to better understand pathological conditions and mechanisms in vivo. Cell-secretome therapies offer a promising cell-free approach to restore musculoskeletal injuries from trauma, infection, tumor, or other musculoskeletal diseases. Improved study and control of the secretome could also lead to the development of new therapeutics and discovery of biomarkers for cancer and other diseases like Type II diabetes. Secretome analysis could also be useful to study disease progression from healthy cells to the metastatic state in different types of cancer, including breast and lung cancers.

Mesenchymal stem cells (MSCs) are multipotent stem cells that can divide and differentiate into a variety of cell types, including osteoblast, chondrocytes, neurons, muscle cells, and adipocytes. MSCs are considered immune-privileged and useful for transplantation and have shown therapeutic efficacy in treating musculoskeletal injuries, improving cardiac function in cardiovascular disease, treating inflammatory disease, and ameliorating the severity of graft versus host disease. Thus, culture and expansion of large numbers of mesenchymal stem cells have been undertaken. However, several drawbacks of MSC use exist including high capital investment, costly cell culture, complicated safety, and quality management issues concerning cell handling, as well as patient discomfort with the invasive procedure necessary for cell collection. Moreover, the survival duration of implanted MSCs is short, and post-transplantation disappearance is noted after several weeks.

Due to the potential of stem cells and secretome production for therapeutic applications, strategies have been proposed to enhance the therapeutic capacity of stem cells (e.g., 3D culture, pharmacological compounds, inflammatory cytokines, and hypoxia). For instance, studies suggest that hypoxia promotes self-renewal of undifferentiated stem cells, increases proliferation rate and cell survival, can accelerate the wound healing process, enhances therapeutic potential, and improves cryoprotective effects. Alterations in local oxygen tension can be translated into a change in cell phenotype through numerous mechanisms, but the hypoxia-inducible factor (HIF) family of transcription factors is often referred to as the master regulator of this process. It has been recognized that the angiogenic potential of endothelial cells is enhanced when stimulated under hypoxic conditions through inducement of expression of angiogenic signaling proteins. However, it is not clear to what extent such changes in the environmental niche affect the MSC proteome. Studies have used chemokines and growth factors to improve stem cell differentiation and therapeutic potency in pulmonary artery hypertension and in the wound healing process. MSC precondition media using inflammatory cytokines has also been shown to improve the immunomodulatory abilities of stem cells, helping to promote tissue regeneration in wound closure model.

While the above describes improvement in the art, room for further improvement exists. For instance, what is needed in the art are methods and materials for modulating the secretome of cells, both in production levels generally and in composition. Methods and materials that can be used to control the secretome of MSCs would be of particular benefit in the art.

### SUMMARY

According to one embodiment, disclosed is a method for culturing cells, and in one particular embodiment, for culturing mesenchymal stem cells (MSCs), and thereby controlling the secretome of the cells. A method can include combining a cell population with a media that includes a catalyst compound, which can include a stilbene or a nicotinamide compound. The method can include culturing the cells in media that includes multiple compounds selected from stilbenes and nicotinamide compounds (e.g., both resveratrol and nicotinamide), simultaneously or sequentially; for instance, a first media that includes a first catalyst compound followed by a second media that includes a second, different catalyst compound. In one embodiment, the catalyst compound(s) can be plant-derived, natural compounds.

Also disclosed is a composition that includes a secretome product in conjunction with at least one catalyst compound as described. For instance, a composition can include an MSC secretome (or a portion thereof) in conjunction with at least one stilbene or at least one nicotinamide compound.

A secretome product as described can be utilized as a cell culture media; for instance, in cell study, biomarker discovery, or the like, or can be utilized in therapeutic applications; for instance, in tissue regeneration, wound healing, angiogenesis, osteogenesis, or treatment of inflammatory response.

### BRIEF DESCRIPTION OF THE FIGURES

A full and enabling disclosure of the present subject matter, including the best mode thereof to one of ordinary skill in the art, is set forth more particularly in the remainder of the specification, including reference to the accompanying figures in which:

FIG. 1 graphically presents the effect of resveratrol (RSV) concentration on MSC proliferation.

FIG. 2 graphically presents the effect of nicotinamide (NAM) concentration on MSC proliferation.

FIG. 3 graphically presents the effect of a combination of RSV and NAM on MSC proliferation.

FIG. 4 graphically presents the protein concentration (μg/ml) using BCA assay in a hydrophilized secretome when samples were treated with a vehicle, dimethyl sulfixide (DMSO) (control), resveratrol (RSV) and resveratrol+nicotinamide (RSV/NAM) under normoxia and hypoxia.

FIG. 5 graphically presents the ratio of the protein concentration for each sample vs control (DMSO under normoxia).

FIG. 6 provides a heat map showing hierarchical clustering of all proteins in the secretome for all samples treated and non-treated. Left-normoxia and right-hypoxia.

FIG. 7 provides a functional annotation of differentily secreted proteins by RSV and RSV+NAM treatment under normoxia and hypoxia as detected by LC-MS/MS in terms of biological processes and specific functions.

FIG. 8 is a table showing differently secreted proteins of an MSC secretome under normoxia compared with a control.

FIG. 9 provides the relative expression (fold change) under nomoxia of important genes in wound healing that were up-regulated (≥1.5 fold) or down-regulated (≤0.5 fold).

FIG. 10 provides the relative expression (fold change) under hypoxia of important genes in wound healing that were up-regulated (≥1.5 fold) or down-regulated (≤0.5 fold).

FIG. 11 provides the relative expression (fold change) under nomoxia of important genes in osteogenesis that were up-regulated (≥1.5 fold) or down-regulated (≤0.5 fold).

FIG. 12 provides the relative expression (fold change) under hypoxia of important genes in osteogenesis that were up-regulated (≥1.5 fold) or down-regulated (≤0.5 fold).

FIG. 13 provides the relative expression (fold change) under nomoxia of important genes in inflammatory response that were up-regulated (≥1.5 fold) or down-regulated (≤0.5 fold).

FIG. 14 provides the relative expression (fold change) under hypoxia of important genes in inflammatory response that were up-regulated (≥1.5 fold) or down-regulated (≤0.5 fold).

FIG. 15 provides the relative expression (fold change) under nomoxia of important genes in angiogenesis that were up-regulated (≥1.5 fold) or down-regulated (≤0.5 fold).

FIG. 16 provides the relative expression (fold change) under hypoxia of important genes in angiogenesis that were up-regulated (≥1.5 fold) or down-regulated (≤0.5 fold).

FIG. 17 grahically illustrates the impact of MSC secretome induced by RSV and RSV+NAM on MSC cell proliferation using a concentration equal to 40 mg/ml under normoxia conditions.

FIG. 18 grahically illustrates the impact of MSC secretome induced by RSV and RSV+NAM on THP-1 Macrophage cell proliferation using a concentration equal to 40 mg/ml under normoxia conditions.

FIG. 19 grahically illustrates the impact of MSC secretome induced by RSV and RSV+NAM on MSC cell proliferation using a concentration equal to 40 mg/ml under hypoxia conditions.

FIG. 20 grahically illustrates the impact of MSC secretome induced by RSV and RSV+NAM on THP-1 Macrophage cell proliferation using a concentration equal to 40 mg/ml under hypoxia conditions.

FIG. 21 presents results of tube formation among human umbilical vein endothelial cells (HUVECs) after 8 hours of incubation on growth factor reduced BD matrigel following treatment with secretome alone, RSV-catalyzed secretome and RSV/NAM-catalyzed secretome under hypoxia.

FIG. 22 presents results of tube formation among HUVECs after 8 hours of incubation on growth factor reduced BD matrigel following treatment with secretome alone, RSV-catalyzed secretome and RSV/NAM-catalyzed secretome under normoxia.

Repeat use of reference characters in the present specification and drawings is intended to represent the same or analogous features or elements of the present invention.

### DETAILED DESCRIPTION

Reference will now be made in detail to various embodiments of the disclosed subject matter, one or more examples of which are set forth below. Each embodiment is provided by way of explanation of the subject matter, not limitation thereof. In fact, it will be apparent to those skilled in the art that various modifications and variations may be made in the present disclosure without departing from the scope or spirit of the subject matter. For instance, features illustrated or described as part of one embodiment, may be used in another embodiment to yield a still further embodiment.

According to one embodiment, described are methods and materials for the production of compositions derived from cell secretome. For instance, in one embodiment, the methods can be utilized to produce conditioned media containing desired proteins of the secretome in controlled amounts.

In general, disclosed methods include culturing cells (e.g., stem cells and MSCs in one particular embodiment) in the presence of one or more catalytic compounds under controlled oxygen content conditions on a surface in a suitable growth media (e.g., 2- or 3-dimensional cell culture). The control of secretome content is affected through the use of one or more compounds as secretome-producing catalyst in conjunction with control of the oxygen content of the culturing environment. Following initial production, the secretome can be processed, for instance via filtration and lyophilization, to provide a powdered product containing proteins, growth factor, cell signals, and other molecules secreted by stem cells. In one embodiment, the secretome product, either as-obtained or following processing, can be further processed to isolate one or more compounds of the secretome product.

In one embodiment, the cells utilized to produce the secretome can be stem cells, and in one particular embodiment, can be MSCs. In such an embodiment, MSCs can be cultured to secrete factors into the culture medium, endowing the medium with potentially useful properties that can be applicable in a variety of applications. For instance, a secretome product obtained as described herein can be useful as a supplement to cell culture, as a therapeutic composition or source thereof, or as a potential economic adjunct or alternative to use of the stem cells themselves. As such, disclosed methods can provide a route for development of cost-effective and shelf-stable compositions that can be used in place of current protocols such as regenerative therapies that require the use of the stem cells themselves.

In one embodiment, secretome produced according to disclosed methods can be utilized to better understand the content of the cell secretome and development of the cell. For instance, challenges in the study of the MSC secretome include the difficulty of collection and preparation of small quantities of secreted proteins and the analysis of the vast number of molecules that comprise the stem cell secretome, as well as the effects of culture modification on the secretome content. Through disclosed methods, particular cells of interest can be catalyzed to produce secretome, which can facilitate better understanding of the secretome, as well as the cells themselves; for instance, providing better understanding of secreted proteins of a particular cell type.

An increasing number of studies show the positive effect of utilizing protein-containing secretome derived from various stem cells in tissue regeneration. These studies confirm the hypothesis of using paracrine/autocrine factors secreted by cells rather than the cells themselves to affect tissue repair. Disclosed methods can facilitate such use through secretome production control. For instance, in one embodiment, secretome formed according to disclosed methods can be utilized to generate a therapeutic composition that can affect in vivo tissue regeneration and that can be administered quickly following injury. Such capabilities can limit scar formation and can avoid issues such as tumorigenicity and immune compatibility associated with ex vivo cell culture. For instance, successful vessel growth of cells cultured in culture media of disclosed secretome product (discussed in more detail in the Examples section) indicates upregulation of angiogeneic stimulators in MSC-treated cells, which supports formation of a pro-angigioneic composition comprising a secretome product as described herein to promote wound healing and angiogenesis.

In yet another embodiment, secreted proteins of a secretome product as described herein can serve as a source for biomarker discovery.

Materials useful for catalyzing secretome production of a cell can include a polyhydroxy stilbene and/or a nicotinamide compound. In one embodiment, a catalyst comound can be a natural compound and in one particular embodiment, a catalyst compound can exhibit beneficial efficacy in its own right. For instance, as the secretome product can carry an amount of the catalyst compound(s) in conjunction with the desired proteins, etc. of the secretome, and the catalyst compound(s) can add to the beneficial properties of the secretome product. Moreover, the use of a catalyst compound under various environments (normoxia and hypoxia) can also be used to enhance the final application of the secretome product.

Culturing of the cells of interest with one or more disclosed catalyst compounds (either together or sequentially) can modulate the type and relative amount of proteins secreted by the cells. In one embodiment, the control capability can be used to target the content of the secretome product to a particular final application (e.g., angiogenesis, wound healing, etc.). As such, control of the secretome content as described can improve the therapeutic capacity of the secretome.

Catalyst compounds used in disclosed methods can modify the secretome of cells to secrete proteins, growth factor, cell signals, and other molecules for potential therapeutic applications such as, and without limitation to, treatment of musculoskeletal injuries, improving cardiac function in cardiovascular disease, treatment of inflammatory disease, ameliorating the severity of graft versus host disease, and encouraging soft tissue regeneration (scar reduction).

The catalyst compounds can be either synthetically produced or natural compounds. The use of natural compounds as catalyst compounds for secretome production can be preferred in some embodiments, e.g., those from medicinal plants, as they can provide a robust ability to target numerous pathways simultaneously with a historical record of safe human consumption and benign side effects. In general, formation of a secretome product can include culturing cells of interest in a typical culture medium, e.g., essential medium, alpha (aMEM) that can be a serum-free media, in conjunction with one or more catalyst compounds as described. In one embodiment, the total amount of catalyst compound in the culture media can be from about 0.05 μg/m L to about 50 μg/m L, for instance from about 0.1 μg/m L to about 40 μg/m L, or from about 1 μg/mL to about 10 μg/mL in some embodiments.

In one embodiment, a catalyst compound can include a stilbene described by the following structure:

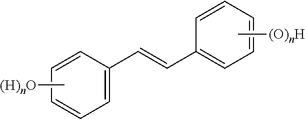

in which each benzene ring can be independently substituted with from 0 to 5 hydroxy moieties.

Exemplary stilbene compounds can include trans-stilbene (1,1′-[(E)-Ethene-1,2-diyl]dibenzene), as well as polyhydroxy stilbenes including, without limitation, resveratrol (3,5,4′-trihydroxy-trans-stilbene), piceatannol (3,5,3′,4′-tetrahydroxy-trans-stilbene), pinosylvin (trans-3,5-dihydroxystilbene), and trans-4-hydroxystilbene.

Resveratrol can be utilized as a catalyst compound in one particular embodiment. Resveratrol is a polyphenolic compound occurring in various plants, including grapes, berries, and peanuts, as a defense mechanism against environmental stresses such as infections and ultraviolet radiation. A remarkable range of biological functions are attributed to resveratrol including cancer prevention, anti-inflammatory, antioxidant activities, and as an anti-obesity compound. It exerts the anti-obesity effect by activating signaling pathways in fat cells that are also activated by energy restriction (i.e., dieting). The result is an increase in lipolysis and fatty acid oxidation that leads to a reduction in body fat. Thus, utilization of resveratrol as a catalyst compound can in some embodiments, provide desirable characteristics to the secretome product, as well as providing for control and desirable qualities in the secretome itself.

In one embodiment, a catalyst compound can include nicotinamide compound, e.g., a nicotinamide or a nicotinamide riboside described by one the following structures:

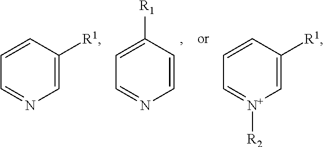

in which
   R₁ is —CONH₂, —CH₂OH, or —COORS
   R₂ is C1 to C5 straight or branched alkyl group, a furanosyl group, a deoxyfuranosl group, or a deoxypryanosyl group; and
   R₃ is a C1 to C24 straight or branched alkyl or alkenyl group.

Exemplary derivatives of the nicotinamide compounds include, without limitation, nicotinic acid esters, nicotinyl amino acids, nicotinyl alcohol esters of carboxylic acids, nicotinic acid N-oxide, niacinamide N-oxide, nicotinyl amino acids (derived, for example, from the reaction of an activated nicotinic acid compound (e.g., nicotinic acid azide or nicotinyl chloride) with an amino acid), and nicotinyl alcohol esters of organic carboxylic acids (e.g., C1-C18). Specific examples of such derivatives include nicotinuric acid and nicotinyl hydroxamic acid and nicotinyl alcohol esters such as nicotinyl alcohol esters of the carboxylic acids salicylic acid, acetic acid, glycolic acid, and palmitic acid, myristic acid, linoleic acid, oleic acid and the like.

Examples of nicotinamide compounds can include, without limitation, nicotinamide, N-methyl nicotinamide, iso-nicotinamide, nicotinamide riboside and mixtures thereof. Other examples of nicotinamide compounds useful herein are 2-chloronicotinamide, 6-methylnicotinamide, N-methyl-nicotinamide, and niaprazine. Nicotinamide compounds used in the present invention may be capable of forming salts, complexes, hydrates, and solvates. Suitable salts are selected from fluoride, chloride, bromide, iodide, formate, acetate, fatty acid carboxylate, fatty acid dicarboxylate, oxalate, ascorbate, benzoate, carbonate, citrate, carbamate, gluconate, lactate, methyl bromide, methyl sulfate, nitrate, phosphate, diphosphate, succinate, sulfate, trifluoroacetate, or trifluoromethanesulfonate.

Nicotinamide furanosides (e.g., nicotinamide riboside) can include derivatives in which the furanosyl group is derived from ribose (i.e., ribofuranos-1-yl) or arabinose, and the deoxyfuranosyl group is derived from deoxyribose. Nicotinamide pyranosides can include derivatives where the pyranosyl group is derived from ribose, arabinose, xylose, lyxose, allose, altrose, glucose, mannose, galactose, gulose, iodose, and talose, and the deoxypyranosyl group is derived from allose, glucose, galactose, and gulose.

In one embodiment, a nicotinamide compound can be a natural compound derived from a plant, e.g., a water-soluble form of vitamin B3 or niacin derived from avocado, peanuts, whole grans, mushrooms, green peas, or potatoes.

In addition to culturing in the presence of one or more catalyst compound(s), a secretome production process can also utilize the oxygen content of the culturing environment to modulate the secretome production and content. For instance, a secretome production process can include culturing the cells of interest under normoxia conditions, e.g., from about 15% to about 25% 02 content, or from about 17% to about 22% 02 content in some embodiments. In some embodiments, a process can include culturing cells of interest under hypoxia conditions, e.g., an oxygen content of less than about 15%, or less than about 10% 02 content in some embodiments; for instance, in an environment including 02 in an amount from about 1% to about 10%, or from about 2% to about 8% in some embodiments.

As an example, and as described further in the Examples section below, MSCs cultured in an environment, including catalyst compounds as described in conjunction with variation in oxygen content during treatment, may not affect overall cell proliferation. However, the total amount of protein in the secretome of cells treated as described can be higher compared with that of a control. Moreover, secretome produced according to disclosed methods can exhibit differentially secreted proteins, and the differentially secreted proteins can be extremely useful in a variety of secretome-based therapy applications such as wound healing, osteogenesis, immune response, treatment of inflammatory-related diseases, and angiogenesis, as well as in topical applications, for instance in cosmetic or skin/hair car applications.

The secretome product of initial culturing methods can be further utilized in a variety of applications including, in one embodiment, as a culture media. In such an application the culturing of cells of interest (e.g., stem cells including MSCs or another cell type of interest) can be carried out at various oxygen content environments. For instance, a secretome product as described, which can include the controlled MSC secretome in conjunction with one or more catalyst comounds, can be utilized as culture media for a cell under normoxia or hypoxia conditions.

Use of a secretome product can enhance culture of cell types of interest. For instance, and as described in further detail herein, MSC secretome catalyzed with disclosed natural compounds can accelerate endothelial cell proliferation, migration, and vascular network formation. A secretome product can be combined with other typical culture media components as desired and can be utilized in culture of any cell type of interest including, without limitation, stem cells (e.g., MSCs), macrophages, endothelial cells, dermal fibroblasts, epidermal keratinocytes, etc.

Following formation, a secretome product can be further processed to isolate one or more components of the product or to produce a dried compound more convenient for storage, shipping, etc. By way of example, in one embodiment, a secretome product can be processed according to an ultrafiltration process, a chromatographic process, etc. Ultrafiltration can be preferred in some embodiments as it is characterized by lower costs, shorter times, and easier scalability for industrial production. Ultrafiltration can also allow for options with regard to a choice of filtration modules with a different molecular weight cut-off as well as retention of the entire secretome. In one embodiment, a secretome product can be further processed according to a lyophilization process, which can provide long-term stability and easy storage and reconstitution of a secretome product for later use.

A secretome product can be utilized in a therapeutic application. In such embodiments, a secretome product can be provided in a pharmaceutical composition and can be administered in any number of forms known to current pharmaceutical practice including, but not limited to, orally, buccally, nasally, rectally, vaginally, topically, transdermally, intravenously, or as an aerosol, an inhalable spray, an injectable solution, a pill, a suppository, a dissolvable powder, a gel capsule, an implanted reservoir, a transdermal patch, a topical cream, or a tincture. A secretome product can further be delivered with any pharmaceutically acceptable carrier, adjuvant, or vehicle.

Pharmaceutically acceptable carriers include, but are not limited to, saline, buffered saline, glucose in saline, etc. Solid supports, liposomes, nanoparticles, microparticles, nanospheres, or microspheres may also be used as carriers for administration of a 7-acetoxyroyleanone. As used herein, the term “pharmaceutically acceptable carrier” is intended to include any and all solvents, solubilizers, fillers, stabilizers, binders, absorbents, bases, buffering agents, lubricants, controlled release vehicles, diluents, emulsifying agents, humectants, lubricants, dispersion media, coatings, antibacterial or antifungal agents, isotonic and absorption delaying agents, and the like, compatible with pharmaceutical administration. The use of such media and agents for pharmaceutically active substances is well-known in the art. Except insofar as any conventional media or agent is incompatible with the active compound, use thereof in the compositions is contemplated. Supplementary agents can also be incorporated into the compositions.

Pharmaceutical compositions for parenteral, intradermal, or subcutaneous injection can include pharmaceutically acceptable sterile aqueous or nonaqueous solutions, dispersions, suspensions, or emulsions, as well as sterile powders for reconstitution into sterile injectable solutions or dispersions just prior to use. Examples of suitable aqueous and nonaqueous carriers, diluents, solvents or vehicles include water, ethanol, polyols (e.g., glycerol, propylene glycol, polyethylene glycol and the like), carboxymethylcellulose and suitable mixtures thereof, vegetable oils (e.g., olive oil), and injectable organic esters such as ethyl oleate. A composition can contain minor amounts of auxiliary substances such as wetting or emulsifying agents, pH buffering agents, and the like that can enhance the effectiveness of the active ingredient. Proper fluidity may be maintained, for example, by the use of coating materials such as lecithin, by the maintenance of the required particle size in the case of dispersions, and by the use of surfactants. A composition may also contain adjuvants such as preservatives, wetting agents, emulsifying agents, and dispersing agents. It may also be desirable to include isotonic agents such as sugars, sodium chloride, and the like.

For intravenous administration, suitable carriers include, without limitation, physiological saline, bacteriostatic water, Cremophor EL™ (BASF, Parsippany, N.J.), or phosphate buffered saline (PBS). In all cases, an injectable composition should be sterile and should be fluid to the extent that easy syringability exists. It must be stable under the conditions of manufacture and storage and must be preserved against the contaminating action of microorganisms such as bacteria and fungi. Prevention of the action of microorganisms may be ensured by the inclusion of various antibacterial and antifungal agents such as paraben, chlorobutanol, phenol, sorbic acid, and the like. Prolonged absorption of the injectable compositions can be brought about by including in the composition an agent that delays absorption, for example, aluminum monostearate or gelatin.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed.

Pharmaceutically compatible binding agents and/or adjuvant materials can be included as part of an orally ingestible composition. The tablets, pills, capsules, troches, and the like can contain any of the following ingredients, or compounds of a similar nature: a binder such as microcrystalline cellulose, gum tragacanth or gelatin; an excipient such as starch or lactose; a disintegrating agent such as alginic acid, Primogel®, or corn starch; a lubricant such as magnesium stearate or Stertes; a glidant such as colloidal silicon dioxide; a sweetening agent such as sucrose or saccharin; or a flavoring agent such as peppermint, methyl salicylate, or orange flavoring.

When administered orally in liquid form, a liquid carrier such as water, petroleum, oils of animal or plant origin such as peanut oil, mineral oil, soybean oil, or sesame oil, or synthetic oils may be added. A liquid form may further contain physiological saline solution, dextrose or other saccharide solution, or glycols such as ethylene glycol, propylene glycol, or polyethylene glycol. When administered in liquid form, a composition can contain from about 0.5 to 90% by weight of a 7-acetoxyroyleanone (or a mixture thereof), in one embodiment from about 1 to 50% by weight of a 7-acetoxyroyleanone.

For administration by inhalation, a pharmaceutical composition can be delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the pharmaceutical compositions are formulated into ointments, salves, gels, or creams as generally known in the art.

In one embodiment, a secretome product can be utilized in place of use of MSCs as used in previously known therapies. The use of secretome rather than the MSCs themselves can decrease the risk of unwanted reactions in allogenic use. Moreover, the use of cell-free therapies in regenerative medicine can provide several advantages over more conventional stem-cell based applications. The use of a secretome product as described can also bypass issues related to immune compatibility, tumorigenicity, and the transmission of infections associated with cell therapies. Secretome product as described can also significantly reduce the time and cost associated with the expansion and maintenance of clonal cell lines since secretome therapies could be prepared in advance in large quantities in an allogeneic or off-the-shelf fashion and be immediately available for treatment when desired, enabling their application to acute conditions such as myocardial infarction, cerebral ischemia, or trauma. Furthermore, the protein milieu delivered for therapeutic application can be tailored to enhance or reduce specific cell-specific effects to produce different therapeutic outcomes using the same initial protein mix.

The present invention may be better understood with reference to the Example, set forth below.

### Example 1

### Materials and Methods

### Cell Culture

Human mesenchymal stem cells (hMSC) were obtained from American Type Culture Collection (ATCC, Manassas, Va.). Cells were stored in liquid nitrogen until use. MSCs were maintained in minimum essential medium, alpha (aMEM, Corning®, Corning, N.Y.) supplemented with 16% Fetal Bovine Serum (FBS, VWR, Radnor, Pa.) and 4 mM L-glutamine (Sigma-Aldrich, St. Louis, Mo.). Medium was changed every other day, and once the cells reached 70-80% confluence, the cells were detached by trypsin-EDTA solution (VWR, Radnor, Pa.). hMSC were seeded (density 5,000 cells/cm²) on 25 cm². When cells reached 80% confluency (4-5 days), they were washed two times with 5 ml of PBS. The growth media was then changed to serum-free media with or without a product treatment (5 ml), and the cells were cultured for 48 hours. Experiments were performed in two different environments, normoxia (about 18.6% oxygen) and hypoxia (5% oxygen). Cells were incubated at 37° C. and 5% CO₂ throughout the experiment.

### Secretome Collection

To characterize the hMSC secretome, condition media (CM or secretome) was collected from cells cultured under FBS-free for 48 hours. CM was filtered through a 0.45 μm syringe filter to remove residual cells and cell debris. The resulting condition media was concentrated and washed 3 times with 50 mM ammonium bicarbonate using Corning® Spin-X® UF Concentrators (10K Dalton, Corning®, Corning, N.Y.) at 3,000 g for 25 minutes each cycle. Following, the secretome was collected from the concentrator, frozen at −80° C., and lyophilized 24 hours. Lyophilized secretome was stored at −80° C. For functional experiments with CM, normal hMSCs (passages 6-8) were used.

### Cytotoxicity Assay

Cell proliferation was assessed using CellTiter 96® AQueous Non-Radioactive Cell Proliferation Assay (MTS, Promega, Madison Wis.) based on the redox conversion of a tetrazolium salt into formazan product. After the CM was removed from the 25 cm², hMSC were washed 2 times with warm PBS, and serum-free media containing 20% MTS solution was added to the cells. The cells were incubated for 2 hours, and the absorbance of each well at 490 nm was measured using a SpectraMax® 190 Microplate Reader.

### Determine Protein Concentration (BCA Assay)

Lyodophylized secretome was dissolved in 50 μl of d-water, and total protein was quantified by bicinchoninic acid assay protein assay (BCA, Cell Signaling Technology, Danvers, Mass.) following manufacturer protocol. Briefly, working reconstitution buffer was diluted 1:1 in d-water, 100 μl of working reconstitution buffer were added to the compatible reagent, and the compatible reagent solution was pipetted up and down 15-20 times. To measure the total amount of proteins in the secretome, 4.5 μl of each sample were pipetted in the center of a 96-well plate, then 2 μl of compatible reagent solution was added in each well. Samples were mixed for 1 minute and incubated in the dark at 37° C. for 15 minutes. Then 130 μl of working reagent was added in each well, mixed for 1 minute and incubated at 37° C. for 30 minutes. Finally, the samples were cooled for 5 minutes at room temperature, and the absorbance of each well was determined at 562 nm using a SpectraMax® 190 Microplate Reader. Standard curves were generated using bovine serum albumin (BSA) standards to obtain the total protein content of each sample.

### Protein Digestion and Mass Spectrometry Analysis (LC-MS/MS)

The amount of secretome was normalized in all samples, and 50 μl of the sample were pipetted in microcentrifuge units (Millipore, Burlington, Mass.) and digested following the filter aided sample preparation (FASP) protein digestion kit from Expedeon (San Diego, Calif.). Samples were reduced using 0.1M dithiothreitol for 15 minutes at 50° C. Alkalization was achieved using iodoacetamide for 20 minutes at room temperature following by 2× washing steps with 8 M urea and 50 mM ammonium bicarbonate. The samples were then enzymatically digested with trypsin at 37° C. overnight. Finally, samples were collected by spinning the microcentrifuge units at 14000 rcf for 20 minutes with a final wash with distilled water and freeze-dried. Prior to LC-MS/MS analysis, samples were resuspended in 0.1% formic acid. Mass spectrometry analysis was executed in the orbitrap Thermo Velos Pro system (Thermo Fisher Scientific, Waltman, Mass.) using Thermo Xcalibur™ Roadmap software (Thermo Fisher Scientific, Waltman, Mass.). For this study, secretome from 4 independent experiments were processed and analyzed independently.

Mass spectra were analyzed using Proteome Discoverer™ 2.1 (Thermo Fisher Scientific, Waltman, Mass.) label-free differential protein expression analysis and the Sequest™ HT. Xcalibur™ raw data collected from the mass spectrometer was imported from the MS spectra and processed as followed. Only features compromising charges of 2+ and 3+ were selected. The mass precision was 2 ppm and mass tolerance 0.02 Da for fragment mass tolerance. Trypsin was set as the digestion enzyme with a maximum of two allowed missed cleavages with six as the minimum peptide length. Cysteine carbamidomethylation (C) was set as a fixed modification, and N-terminal acetylation and methionine oxidation were allowed as variable modifications. The spectra were searched using the Sequest™ HT search engine against the human Uniprot sequence database. Percolator node was used to filter data using 0.1 for FDR at the peptide and protein level. For relative quantification in a label-free analysis, the precursor ion area detector node was used, and ratios were calculated by dividing the treated samples vs. the control group (DMSO under normoxia). The Database for Annotation, Panther 14.1, was used to assign Gene Ontology (GO) annotations biological process, and the Proteome Discoverer™ 2.2 was used to find specific functions such as wound healing, osteogenesis, inflammatory response, and angiogenesis.

Therapeutic Potential of hMSC Secretome

Human monocytic cells (THP-1 cells) were obtained from the American Type Culture Collection (ATCC), and cultured in RPMI-1640 media supplemented with 10% fetal bovine serum (FBS), and 0.05 mM 2-mercaptoethanol. THP-1 cells were differentiated into MO macrophages by culturing the cells with 100 ng/ml of 12-myristate 13-acetate (PMA) (Sigma) for 24 hours. After differentiation, the cells were washed to remove non-differentiated cells.

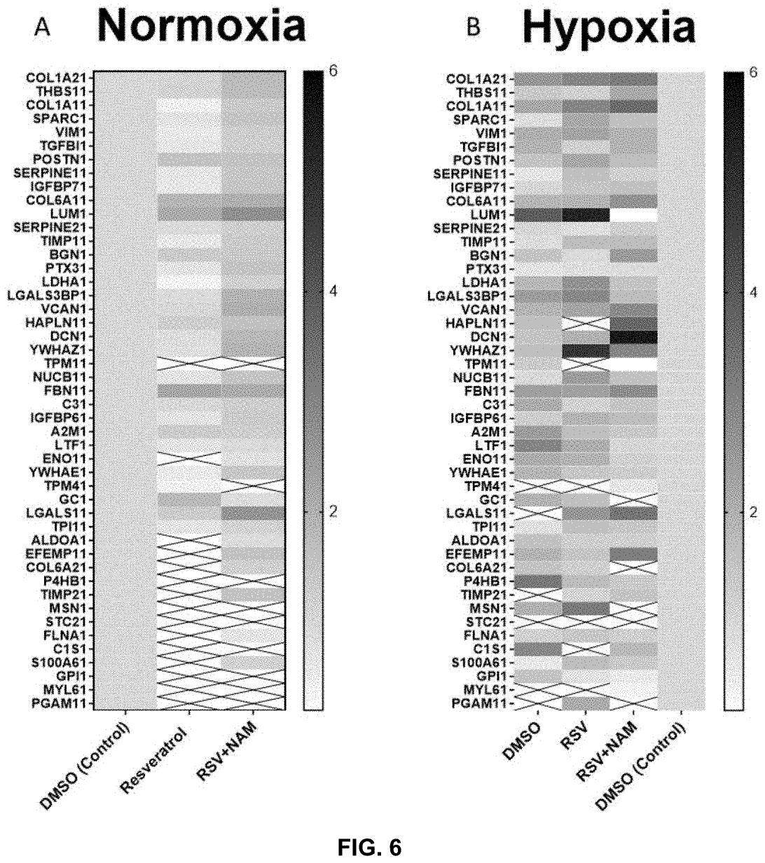

To assess the effect of secretome on cell growth, MSCs and macrophages were seeded in 96-well plates and allowed to adhere for 24 hours. Then, different concentrations of secretome (0, 0.1, 1, 10, 20, and 40 μg/ml) in growth media was added to the cells followed by incubation for 48 hours and cell proliferation was monitored using the MTS assay (FIG. 6 A-D).

An in vitro tube formation assay was performed to study the effect of MSC secretome on angiogenesis. Growth factor reduced BD Matrigel™ (BD Biosciences, Franklin Lakes, N.J.) was used for this experiment. 50 μL of Matrigel™ were added to each well of a pre-chilled 96-well plate and incubated at 37° C. and 5% CO₂ for 30 minutes until the Matrigel™ gelled. A suspension of 20,000 human umbilical vein endothelial cells (HUVECs) in 100 μL of cell culture media supplemented with catalyzed secretome at different concentrations was added on top of the gel and incubated at 37° C. for 8 hours. The control group was treated only with secretome DMSO treated cells. Following the 8 hour incubation time point, the cells were fixed and stained using staining solution. The number of tubes was examined using a phase-contrast inverted microscope (Invitrogen™ EVOS™ FL Auto Cell Imaging).

### Results

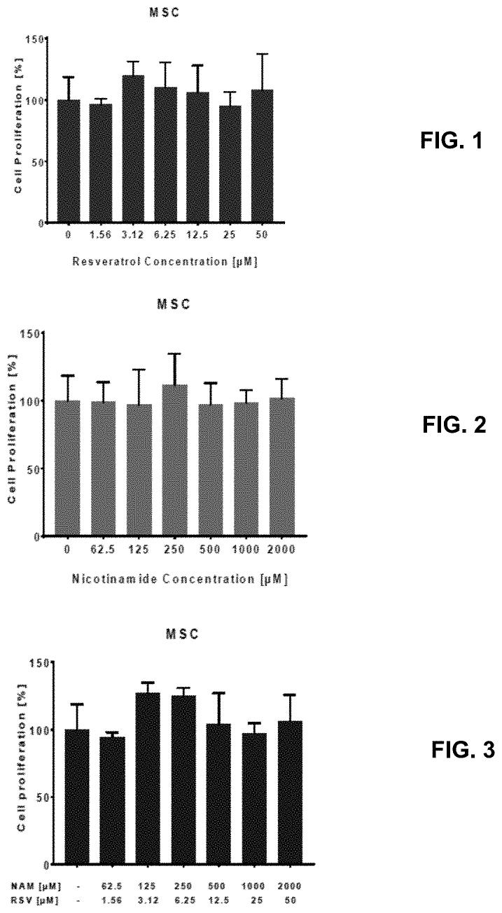

Cell proliferation was determined using MTS, colorimetric assay after the cells were treated for 48 hours in serum free media with the vehicle (DMSO) as control, resveratrol (RSV), nicotinamide (NAM), or a combination (RSV+NAM) in serum-free media under normoxia. The results showed cell proliferations change with the increasing dose of resveratrol (FIG. 1), nicotinamide (FIG. 2), and RSV+NAM (FIG. 3). The results showed that there is not a significant difference between the groups when compared with the control. Results were represented as percentage ±SD. *p<0.05, t-test versus control group. No significant changes were observed in cell number when exposed to these compounds.

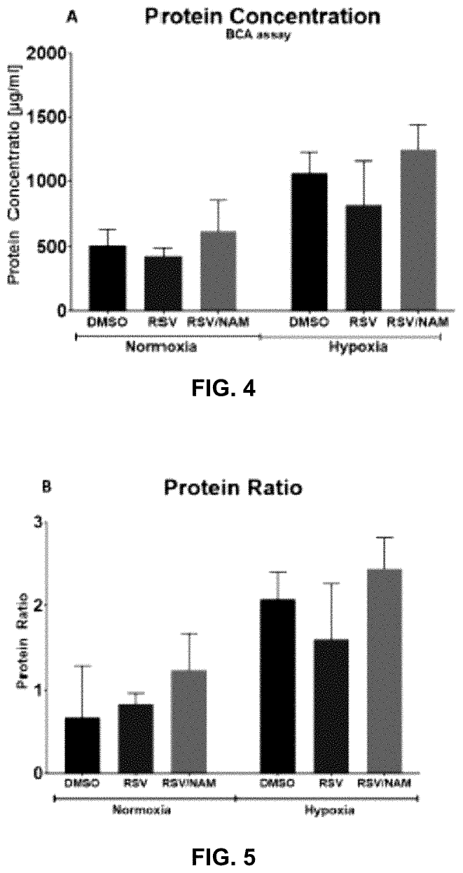

Secretome protein concentration was measured using the BCA protein assay kit. GraphPad Prism 7 was used to determine the total protein concentration (μg/ml). Following, protein concentration measurements for all groups were divided by the fraction of cell proliferation assay to determine the relative amount of protein per flask (n=4). FIG. 4 shows the total protein concentration in each group after the secretome was lyophilized and resuspended in d-water. As shown, the amount of protein for all groups that were cultured under hypoxia were higher than those in normoxia. The ratio for all groups was calculated vs. the control group (DMSO under normoxia), and the higher amount of protein under normoxia and hypoxia was found to be 1.22 and 2.44 fold for the RSV/NAM treated group, respectively (FIG. 5).

LC-MS/MS analysis was performed to identify secreted proteins in the secretome from treated and non-treated cells under normoxia and hypoxia (5% O₂) conditions. Table 1, below, shows the number of proteins secreted (n=4), the number of unique proteins, and the number of up-regulated (fold ≥1.5) and down-regulated (fold ≤0.5) proteins in each group when compared with the control group (cells exposed to the vehicle, DMSO, under normoxia condition).

TABLE 1

| | Normoxia | | | Hypoxia | | |
|---|---|---|---|---|---|---|
| | | | RSV + | | | RSV + |
| | DMSO | RSV | NAM | DMSO | RSV | NAM |
| Fold ≥1.5 | — | 7 | 30 | 39 | 30 | 27 |
| Fold ≤1.5 | — | 3 | 0 | 1 | 0 | 3 |
| Unique vs | — | 2 | 2 | 8 | 2 | 2 |
| control | | | | | | |
| Total | 61 | 51 | 61 | 59 | 55 | 60 |
| proteins | | | | | | |

A subset of proteins whose levels changed more than 1.5-fold for up-regulated proteins or 0.5-fold for down-regulated proteins was found in the secretome in all treatment conditions (FIG. 6) under normoxia (left) and hypoxia (right). The clustering of this subset of proteins showing expression changes indicate samples from the secretome are entirely separated. The shade in each cell of the heatmaps of FIG. 6 indicates the protein expression ratio as compared to the control (DMSO under normoxia) from white for the largest decrease to black for the largest increase on the average expression fold change across all stimulations. Cells with an X represent missing data. Each row is a protein and each column is a sample. Samples are name based on data type, and treatment.

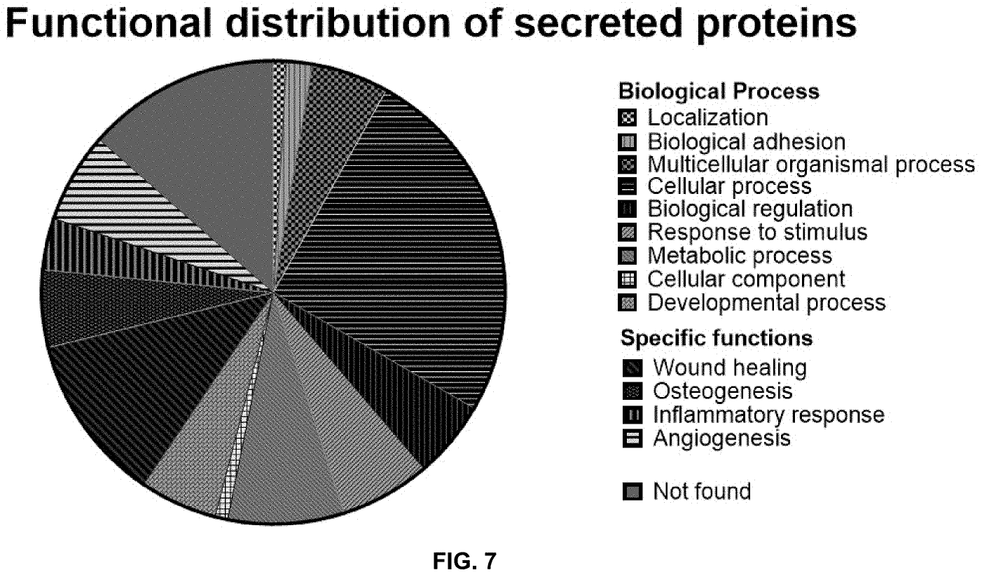

To determine the potential function of the dysregulated secreted proteins induced by RSV or RSV+NAM, under normoxia or hypoxia, gene ontology analysis was performed by integrating the results obtained in Proteome Discoverer™ 2.2. Significant over-representation was found of categories mainly related to the biological process and specific functions, specifically, wound healing, osteogenesis, inflammatory response, and angiogenesis (FIG. 7). The differentially secreted proteins by RSV and RSV+NAM under normoxia and hypoxia detected by LC-MS/MS were interrogated in terms of functional annotation by Panther 14.1 (biological process), and the specific functions were found by using Proteome Discoverer™ 2.2.

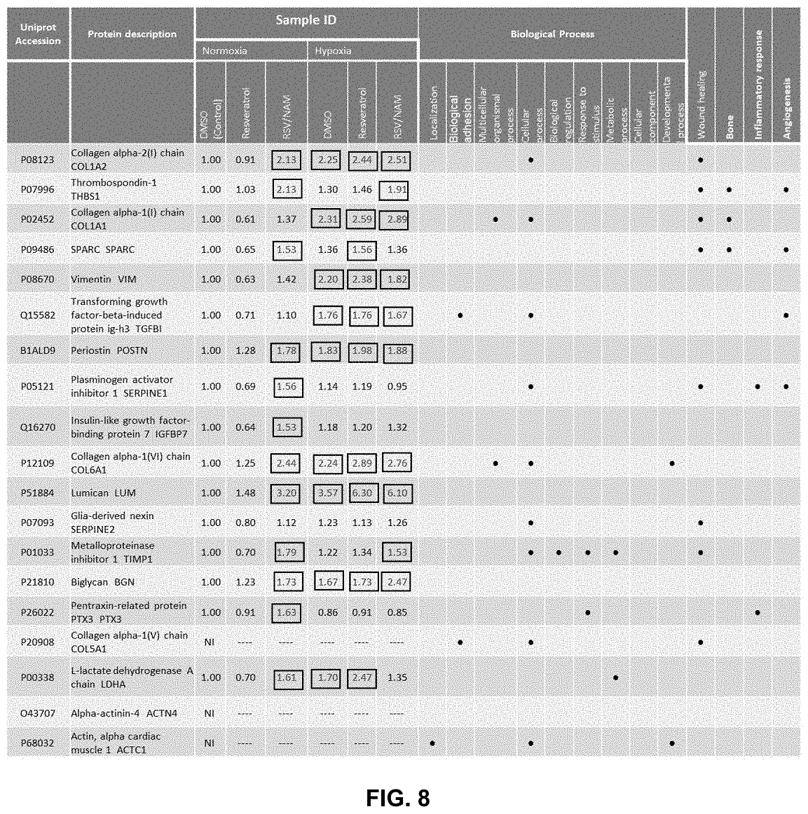

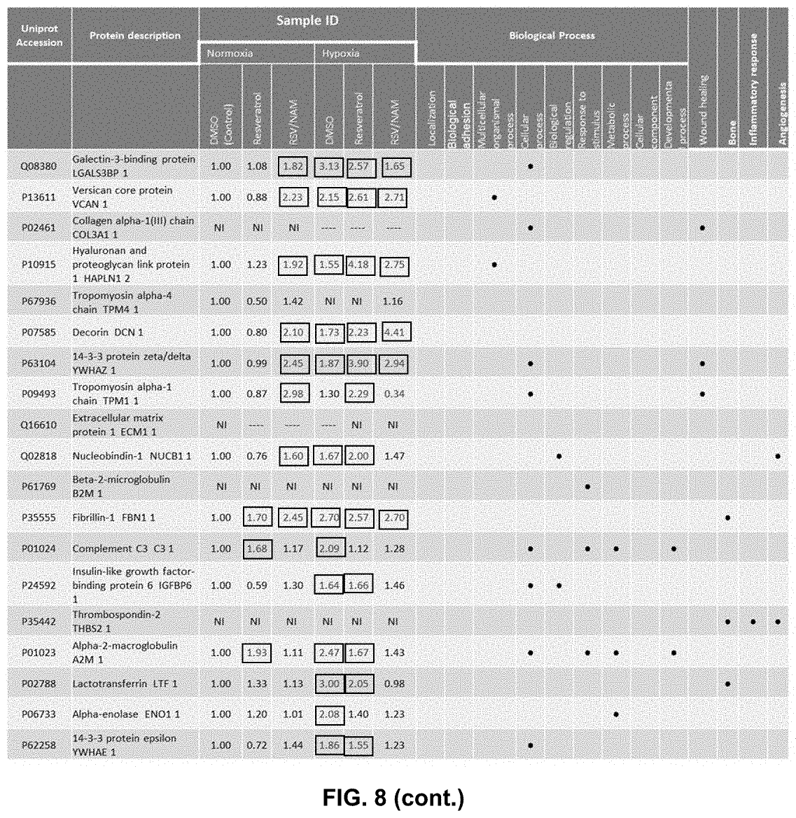

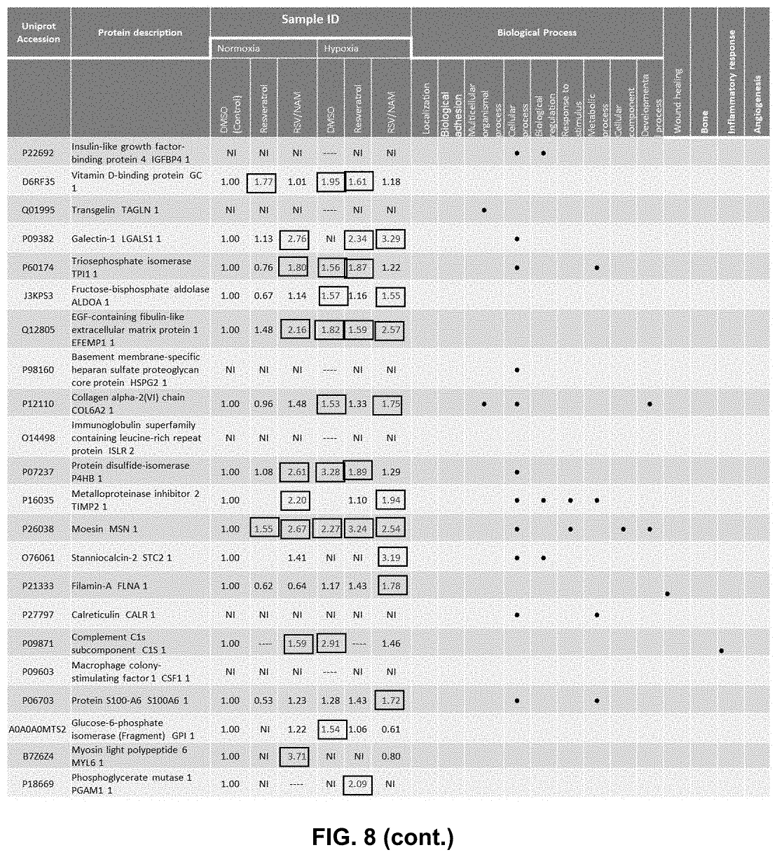

FIG. 8 is a table showing all differentially secreted proteins in the treated MSC secretome compared with DMSO (control) under normoxia (fold change). Numbers in squares indicate the up-regulated proteins 1.5 fold change while numbers not in a square indicate the down-regulated proteins 0.5 fold change. Proteins that were not found or identify (NI) in the control group did not calculate as a fold change (---). The black points indicate the Gene Ontology annotations according to their biological function and specific functions (wound healing, bone, inflammatory response, and angiogenesis).

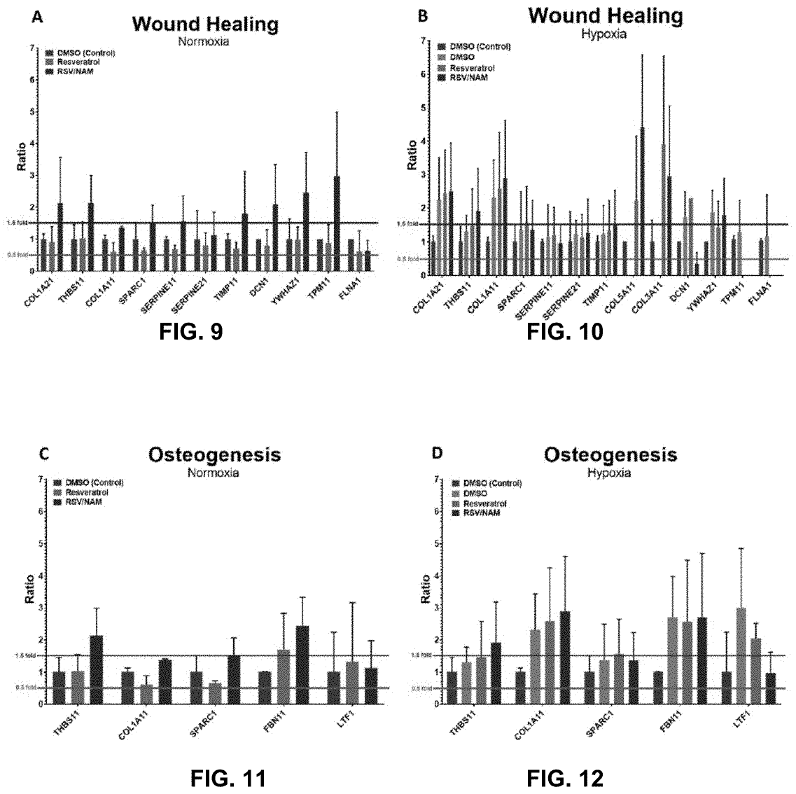

FIG. 9 through FIG. 16 show the relative expression of essential genes for wound healing (FIG. 9, normoxia; FIG. 10, hypoxia), osteogenesis (FIG. 11, normoxia;

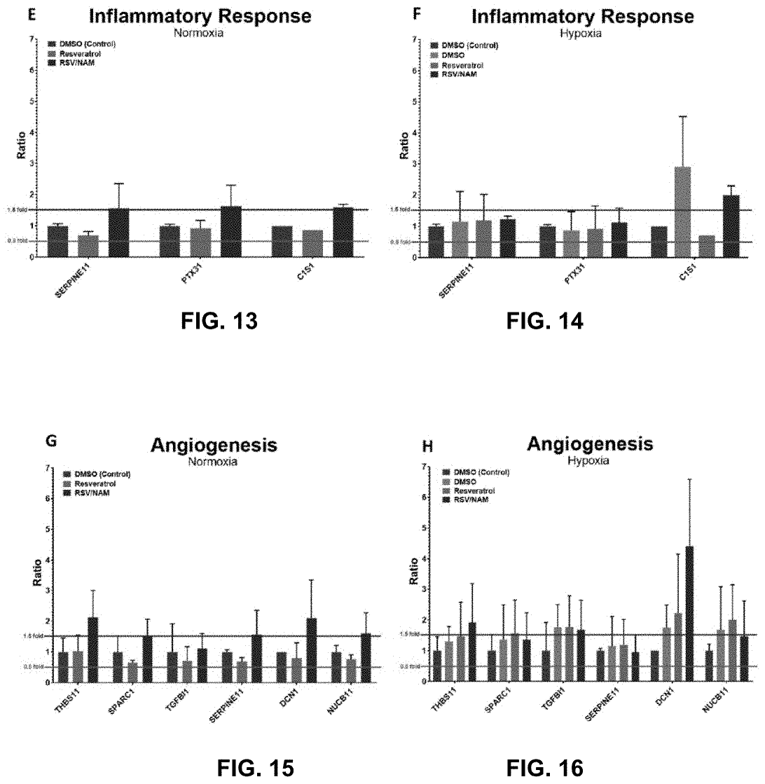

FIG. 12, hypoxia), immune response (FIG. 13, normoxia; FIG. 14, hypoxia), and angiogenesis (FIG. 15, normoxia; FIG. 16, hypoxia) in the secretome for the different groups under normoxia and hypoxia when compared with the control group (DMSO under normoxia).

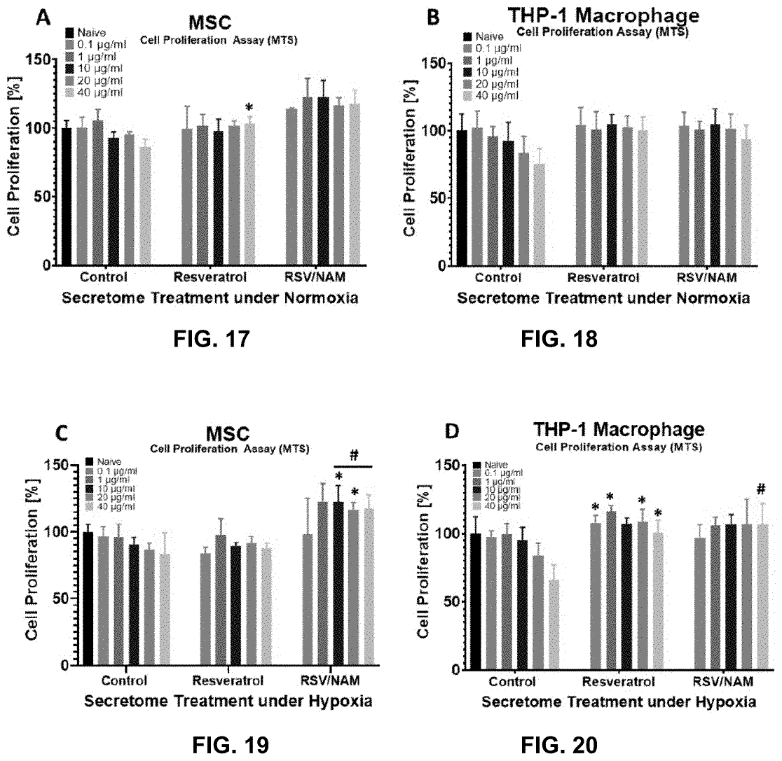

Tissue regeneration and homeostasis rely on cell proliferation, stem cell differentiation, immunomodulation, cellular migration, and vascularization. Thus, the impact of the MSC secretome on cell proliferation was investigated using MSC and THP-1 derived macrophages. MTS assay of the MSC exposed for 48 hours to the total RSV-catalyzed secretome revealed a significant increase of cell proliferation compared to control cells when then the concentration of the secretome was 40 μg/ml (FIG. 17), but the results were not a strong for the macrophage (FIG. 18). Moreover, the total RSV-catalyzed secretome and RSV/NAM-catalyzed under hypoxia was able to stimulate the MSC and THP-1 macrophages proliferation at various concentrations when compared with the control group (FIG. 19, FIG. 20). As indicated, results were more significant when cells were treated with RSV-catalyzed secretom of RSV/NAM-catalyzed secretome under hypoxia condition (FIG. 19, FIG. 20) when compared with the control group (secretome without any compound). On the Figures, * represents a significant difference (p 0.05) from the control group, which was calculated using GraphPad Prism 7.03 to determine the significance between each experimental group and control and # represents a significant difference (p 0.05) from resveratrol and RSV/NAM groups.

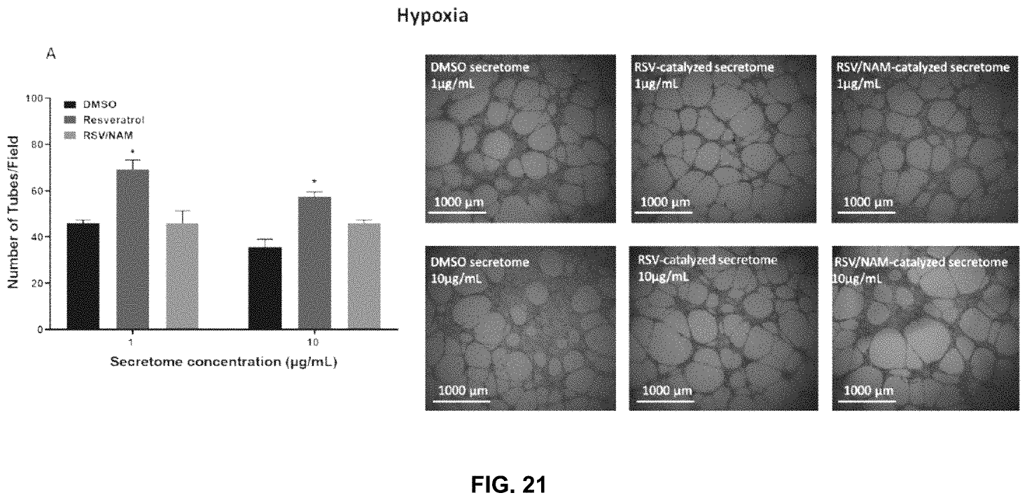

The potential stimulatory effect of RSV-catalyzed secretome and RSV/NAM-catalyzed secretome was assessed under hypoxia and normoxia using in vitro tube formation assay using HUVECs. As shown in FIG. 21 under hypoxia, RSV-catalyzed secretome at different concentrations of 1 and 10 μg/mL demonstrated the highest amount of tubular formation with an organized network as compared to the non-treated cells or secretome DMSO-treated cells. However, RSV/NAM-catalyzed secretome had a similar pattern to the control. An increased concentration of secretome reduced angiogenesis for all the samples.

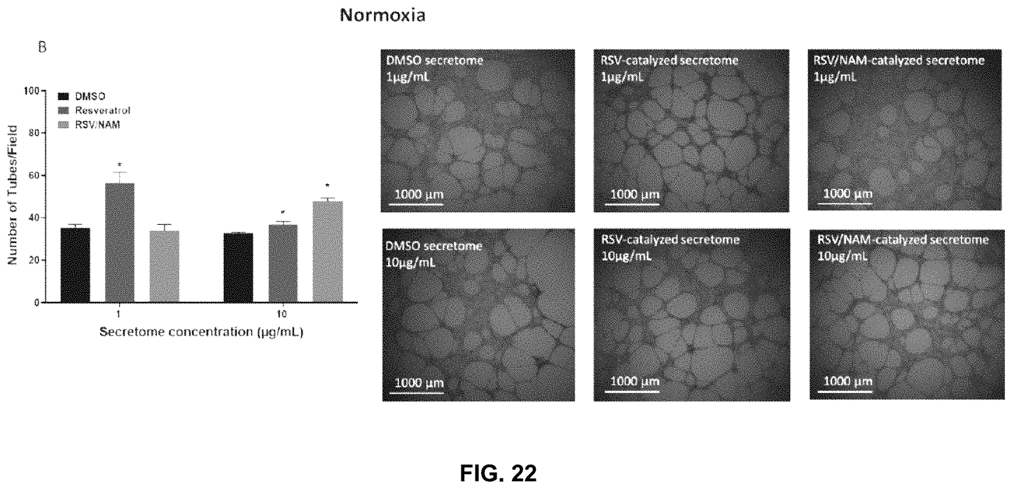

Angiogenesis of the HUVECs under normoxia condition (FIG. 22) showed a significant amount of vascular tube formation for RSV-catalyzed secretome at a concentration of 1 μg/mL as compared to the control and RSV/NAM-catalyzed secretome. However, fewer tubes were formed under normoxia conditioned samples. The significant numbers of vascular tubes formed by RSV-catalyzed secretome under both conditions prove that resveratrol has the potential to induce angiogenic proteins in the secretome, which ultimately affects vascularization. (In FIG. 21 and FIG. 22, * represents a significant difference (p 0.05) from the control group, which was calculated using GraphPad Prism 7.03 to determine the significance between each experimental group and control.)

While certain embodiments of the disclosed subject matter have been described using specific terms, such description is for illustrative purposes only, and it is to be understood that changes and variations may be made without departing from the spirit or scope of the subject matter.

What is claimed is:

1. A method for culturing cells comprising combining a cell population with a first media, the first media comprising a first catalyst compound, the first catalyst compound comprising a stilbene or a nicotinamide compound.

2. The method of claim 1, the first media comprising one or more additional catalyst compounds, the one or more additional catalysts compounds comprising a stilbene or a nicotinamide compound.

3. The method of claim 1, further comprising combining the cell population with a second media, the second media comprising a second catalyst compound that differs from the first catalyst compound, the second catalyst compound comprising a stilbene or a nicotinamide compound.

4. The method of claim 1, the stilbene comprising a polyhydroxy stilbene.

5. The method of claim 4, the polyhydroxy stilbene comprising resveratrol.

6. The method of claim 1, the nicotinamide compound comprising nicotinamide or nicotinamide roboside.

7. The method of claim 1, wherein the first catalyst compound is a plant-derived natural compound.

8. The method of claim 1, the cell population comprising stem cells.

9. The method of claim 1, the stem cells comprising mesenchymal stem cells.

10. A composition comprising a cell secretome and a first catalyst compound, the first catalyst compound comprising a stilbene or a nicotinamide compound.

11. The composition of claim 10, wherein the cell secretome is a mesenchymal stem cell secretome.

12. The composition of claim 10, the cell secretome comprising one or more proteins involved in wound healing, osteogenesis, inflammatory response, or angiogenesis.

13. The composition of claim 10, the composition further comprising one or more additional catalyst compounds, the one or more additional catalysts compounds comprising a stilbene or a nicotinamide compound.

14. The composition of claim 10, the stilbene comprising a polyhydroxy stilbene.

15. The composition of claim 14, the polyhydroxy stilbene comprising resveratrol.

16. The composition of claim 10, the nicotinamide compound comprising nicotinamide or nicotinamide roboside.

17. The composition of claim 10, wherein the first catalyst compound is a plant-derived natural compound.

18. A culture media comprising the composition of claim 10.

19. A pharmaceutical composition comprising the composition of claim 10.
